(19) United States
(12) Patent Application Publication
(10) Pub. No.: US 2023/0170091 A1
(43) Pub. Date: Jun. 1, 2023

(54) AIR Oracle Brain

(71) Applicant: Anuthep Benja-Athon, New York, NY (US)

(72) Inventor: Anuthep Benja-Athon, New York, NY (US)

(73) Assignee: Anuthep Benja-Athon, New York, NY (US)

(21) Appl. No.: 17/535,996

(22) Filed: Nov. 27, 2021

(51) Int. Cl. G16H 50/20; G16H 50/70; G06N 5/04

(52) U.S. Cl. CPC G16H 50/20; G16H 50/70; G06N 5/042; G06N 5/046

(57) ABSTRACT

Interactive and adaptable digital router for rendering diagnoses wherein interactive multi-dimensional figurines rendering diagnoses or reverse deducing diagnoses by rendering diagnoses-related adept networked repositories-rendered Interactive and changeable approximates, similars, accurate, match and same as queries.

This is a continuation-in-part of Ser. No. 15/881,805 entitled Abiotic Intelligence-Rendered Pay filed Jan. 29, 2018, Ser. No. 15/495,164 entitled AIR Oracle filed Apr. 24, 2017 and U.S. Pat. No. 6,132,218 entitled Images for communication of medical information in computer Oct. 17, 2000.

### FIELD OF THE INVENTION

Abiotic intelligent digital router diagnosing diseases and disorders

### BACKGROUND OF THE INVENTION

The status quo healthcare perpetually escalating healthcare and business costs and pain and suffering for billions. $trillion corporations' years of attempts failed to find the solution.

AIR Oracle Brain is the omnipotent and omnipresent abiotic intelligent digital router for diagnosing diseases and disorders never able by any and all human including physicians, technologies, machines and means.

AIR Oracle Brain is far more superior than human brain which can never replicate its abiotic circuitry thus realm and function because AIR Oracle Brain can optimally and rapidly render responses by forward rendering and extrapolating responses based on pathologic basis and clinical basis of disease and, conversely, reverse deduction for rendering responses to queries of data in, between and among networked communication devices described herein.

As a continuation-in-part of said applications, the present invention comprises digital router and compilations of algorithms of relational data complementing said earlier inventions so that billions of networked devices can use said inventions including the AIR Oracle Brain to optimally, rapidly and accurately achieve the goals and objectives set forth in said inventions.

### SUMMARY OF THE INVENTION

The interactive and adaptable digital router comprising abiotic intelligent adept networked repositories for rendering adaptable labelled, accentuated and highlighted multi-dimensional anatomical views, illustrations and medical-imagings of digital figurines wherein said adaptable labeled, accentuated and highlighted adaptably attaching and associating and detaching and dissociating plurality of interactive popups for directly or conversely coordinating, routing and showing queries of data to responses in and among said anatomical views, illustrations and medical-imagings in and among networked communication devices.

### DESCRIPTION OF THE PREFERRED EMBODIMENTS

According to Claim 1 infra, in other words, the AIR Oracle Brain being the interactive and adaptable digital router for autocoordinating and autorouting and showing networked repositories for querying to interactive and adaptable responses (aka responses) to interactive and adaptable queries (aka queries) of said networked repositories, wherein abiotic intelligence for instructing adept networked repositories for rendering a function comprised a plurality of interactive, adaptable and changeable multi-dimensional figurines comprised iLAH and interactive and adaptable popups wherein adept networked repositories for getting and rendering, option completing, autocompleting responses and toggling queries and responses and vice versa in, between and among interactive and adaptable popups and iLAH for attaching and associating, or detaching and dissociating interactive and adaptable popups in, between and among said plurality of interactive, adaptable and changeable multi-dimensional figurines in, between and among said networked communication devices comprised networked servers, smartphones, laptops, desktops and medical-imaging devices and equipment, according to chronologically ordered sequences of relational timestamped data related to iLAH.

Said adept networked repositories for getting and rendering, option completing, autocompleting responses comprised interactive and changeable approximates, similars, accurate, match and same as queries for getting from memories and rendering, option completing, autocompleting adept networked repositories-rendered interactive and changeable diagnoses and related diagnoses and differential diagnoses collectively known herein as interactive and changeable diagnoses.

According to Claim 1 infra, in other words, complementary to said function, abiotic intelligence for instructing said adept networked repositories for rendering reverse deduction for autocoordinating and autorouting and showing a plurality of interactive, adaptable and changeable multi-dimensional figurines comprised iLAH attaching and associating, or detaching and dissociating interactive and adaptable popups comprised interactive and changeable diagnoses and related interactive and adaptable popups comprised diagnoses-related adept networked repositories-rendered interactive and changeable approximates, similars, accurate, match and same as tiny, small, scanty or piecemeal queries including inaccuracies in plurality of popups attaching and associating, or detaching and dissociating, with iLAH, according to chronologically ordered sequences of relational timestamped data related to iLAH.

Further to supra, said omnipotence and omnipresence are said adept networked repositories getting adept networked repositories-rendered bits of data which are scanned, matched and related said bits of data with similar, same and distinguishing chronologically ordered relational sequences of timestamped data in said compilations of relations of genes-processor units-rendered fundamental medicine-health-healthcare relational data to render approximates, similars, accurate, match and same and related interactive and changeable diagnoses and related chronologically ordered sequences of relational timestamped data related to iLAH, according to chronologically ordered sequences of relational timestamped data related to iLAH.

Adept networked repositories for getting and rendering, option completing, autocompleting responses and toggling queries and responses and vice versa in, between and among interactive and adaptable popups and iLAH for attaching and associating, or detaching and dissociating interactive and adaptable popups.

Adept networked repositories for getting and rendering, option completing, autocompleting responses and toggling queries and responses and vice versa in, between and among interactive and adaptable popups and iLAH for attaching and associating, or detaching and dissociating interactive and adaptable popups.

Interactive and changeable diagnoses for getting and rendering, option completing, autocompleting adept networked repositories-rendered interactive and changeable approximates, similars, accurate, match and same as queries and vice versa i.e. adept networked repositories-rendered interactive and changeable approximates, similars, accurate, match and same for getting and rendering, option completing, autocompleting interactive and changeable diagnoses.

Adept networked repositories-rendered interactive and changeable approximates, similars, accurate, match and same comprised adept networked repositories-rendered relation data in said compilations of relations of fundamental medicine-health-healthcare relational data.

iLAH for adaptably attaching and associating, or detaching and dissociating, said plurality of interactive popups=

f(networked repositories-rendered queries, processor units-rendered sequences of timestamped data including images and videos, adept networked repositories-rendered responses, adept networked repositories-rendered chronologically ordered relational sequences of timestamped data including images and videos, renderings, adept networked repositories-rendered Interactive and changeable same, matches, diagnoses, related diagnoses and differential diagnoses)

f(pathologic basis and clinical basis of diseases and disorders).

In other words, the plurality of interactive, adaptable and changeable multi-dimensional figurines comprised iLAH for adaptably attaching and associating, or adaptably detaching to and dissociating, said plurality of interactive popups, according to said interactive, adaptable and changeable queries and responses in and among said plurality of interactive popups or, in other words, according to chronologically ordered sequences of relational timestamped data related to iLAH.

The plurality of interactive, adaptable and changeable multi-dimensional figurines comprising iLAH for adaptably attaching and associating, or adaptably detaching and dissociating, said plurality of interactive popups comprised queries and responses, according to said interactive, adaptable and changeable queries and responses related to iLAH in, between and among said plurality of interactive popups. Said plurality of interactive popups comprised any or any combinations of said words, terms, phrases, images and videos with or without 3D coordinates, chronologically ordered sequences of relational timestamped data, diagnoses and related diagnoses and differential diagnoses, and interactive adaptable visual-hierarchized constituent flowing frameworks (SIVSF) of and silhouetting interactive adaptable universal network data-driven visualizations of visual-hierarchized flowing frameworks (SIUNDV) (Ser. No. 15/495,164 entitled AIR Oracle).

Said connected assortments of SIVSF for silhouetting SIUNDV comprised said words, terms, phrases, images and videos with or without 3D coordinates, chronologically ordered sequences of relational timestamped data, diagnoses and related diagnoses and differential diagnoses.

Said iLAH for adaptably attaching and associating with multi-dimensional human figurines and said medical imagings in, between and among said networked communications devices including said medical-imaging devices and equipment.

and, conversely, adaptably detaching and dissociating a plurality of interactive, adaptable and changeable popups=

f(networked repositories-rendered queries, processor units-rendered sequences of timestamped data including images and videos, adept networked repositories-rendered responses, adept networked repositories-rendered chronologically ordered relational sequences of timestamped data including images and videos, renderings, adept networked repositories-rendered Interactive and changeable same, matches, diagnoses, related diagnoses and differential diagnoses)

f(pathologic basis and clinical basis of diseases and disorders).

Therefore, the permutations of iLAH related queries and responses in and among said multi-dimensional anatomical views, illustrations and medical-imagings of digital figurines are infinite.

Therefore, the permutations of iLAH adaptably attaching and associating and detaching and dissociating said plurality of interactive popups of queries and responses in and among said multi-dimensional anatomical views, illustrations and medical-imagings of digital figurines are infinite.

The iLAH comprised said adept networked repositories and networked repositories-rendered interactive, adaptable and changeable labeled, accentuated and highlighted visual, audio and digital multi-dimensional gross, microscopic and digital anatomical images, medical imagings, views, areas, sites, parts, organs representations and illustrations comprised 3D coordinates, according to said interactive, adaptable and changeable popups comprised said queries and responses in, between and among said viewing screens and touchscreens of said networked communication device.

Said innovative computer programs including thesaurus software for rendering professional and technical terminologies—lay terminologies thesaurus software for instructing said networked repositories to constitute and render relational professional and technical terminologies—lay terminologies analogies such as, but not limited to, English and non-English data in said touchscreens and viewing screens and in said memories. In other words, said thesaurus serves as the languages translator of data in and among said touchscreens and viewing screens wherein said data are displayed in languages, images and videos comprehensible and useable by and among laypeople and professional people. Said innovative computer programs applies said thesaurus in and among networked servers and devices repositories.

So, in response to queries, said innovative computer programs including thesaurus software instructing adept networked repositories scanning, matching and relating said any or any combinations of said words, terms, phrases, images and videos with or without 3D coordinates activating said innovative computer programs including thesaurus software instructing adept networked repositories to getting, organizing, analyzing and organizing said queries in ordered sequences of relational timestamped data and scanning, matching and relating said ordered sequences of relational timestamped data with similar, same and distinguishing chronologically ordered sequences of relational timestamped data, in compilations of relations of genes-processor units-rendered compilations of relations of fundamental medicine-health-healthcare relational data to rendering approximates, similars, accurate, match and same, relating to characteristic iLAH, to said queries being stored in said compilations of relations of fundamental data structure in said memories in, between and among said adept networked repositories and networked repositories.

Furthermore, queries activating said innovative computer programs including thesaurus software instructing adept networked repositories toggling responses in, between and among plurality of interactive popups, toggling responses in plurality of interactive popups in, between and among iLAH;

responses activating said innovative computer programs including thesaurus software instructing adept networked repositories toggling queries in, between and among plurality of interactive popups, toggling queries in plurality of interactive popups in, between and among iLAH; and

queries and responses toggling iLAH in, between and among plurality of interactive, adaptable and changeable multi-dimensional figurines, according to chronologically ordered sequences of relational timestamped data related to iLAH, in, between and among said networked communication devices.

In, between and among plurality of interactive popups comprised responses which are approximates, similars, accurate, match and same for adaptably attaching and associating with characteristic iLAH, according to said chronologically ordered sequences of relational timestamped data related to iLAH in, between and among plurality of interactive, adaptable and changeable multi-dimensional figurines in, between and among said networked communication devices.

Further to supra, in response to queries, said innovative computer programs including thesaurus software instructing adept networked repositories option completing, autocompleting said any or any combinations of said adept networked repositories-rendered Interactive and changeable approximates, similars, accurate, match and same and related to characteristic iLAH in, between and among said plurality of interactive popups adaptably attaching and associating with characteristic iLAH.

Reversibly, adaptably detaching to and dissociating iLAH according to processor units-rendered scans, matches and relations approximates, similars, accurate, match and same and related characteristic iLAH according to adapting, adding, editing, option completing, autocompleting responses and queries in, between and among said plurality of interactive popups adaptably attaching and associating with characteristic iLAH, according to said chronologically ordered sequences of relational timestamped data related to iLAH in, between and among plurality of interactive, adaptable and changeable multi-dimensional figurines in, between and among said networked communication devices.

Furthermore, responses activating said innovative computer programs including thesaurus software instructing adept networked repositories toggling queries in, between and among plurality of interactive popups, toggling responses in plurality of interactive popups in, between and among iLAH;

queries activating said innovative computer programs including thesaurus software instructing adept networked repositories toggling queries in, between and among plurality of interactive popups, toggling queries in plurality of interactive popups in, between and among iLAH; and queries and responses toggling iLAH in, between and among plurality of interactive, adaptable and changeable multi-dimensional figurines,

according to chronologically ordered sequences of relational timestamped data related to iLAH, in, between and among said networked communication devices.

Furthermore, queries for toggling responses in, between and among plurality of interactive popups;

queries for toggling responses in plurality of interactive popups in, between and among iLAH; responses for rendering, option completing, autocompleting queries in, between and among iLAH;

queries;

responses for toggling queries in, between and among plurality of interactive popups; responses for toggling queries in plurality of interactive popups in, between and among iLAH; and

queries and responses for toggling iLAH in, between and among plurality of interactive, adaptable and changeable multi-dimensional figurines in, between and among said networked communication devices.

Therefore, the permutations of iLAH related queries and responses in and among said multi-dimensional anatomical views, illustrations and medical-imagings of digital figurines are infinite.

Therefore, the permutations of iLAH adaptably attaching and associating, or detaching and dissociating, said plurality of interactive popups of queries and responses in and among said multi-dimensional anatomical views, illustrations and medical-imagings of digital figurines are infinite.

In other words, like every diagnosis endowed with a name, interactive and adaptable digital router endowed every diagnosis a unique labeled, accentuated and highlighted multi-dimensional anatomical views, illustrations and medical-imagings comprised said relational data, To further describe one of the preferred embodiments of said first and second functions of said interactive and adaptable digital router for coordinating and routing in and among said interactive, adaptable and changeable labeled, accentuated and highlighted, MRI 3D coordinates—anatomical areas, sites, parts and organs; CTScan 3D coordinates: anatomical areas, sites, parts and organs; x-ray 3D coordinates: anatomical areas, sites, parts and organs; ultrasound 3D coordinates: anatomical areas, sites, parts and organs for showing and rendering queries and responses in, between and among 65 medical and surgical specialties.

CorPulmonaleHeartFailure; CongenitalHeart; Endocarditis; IschemicHeart; ImagingHeart; LaboratoryHeart; PercutaneousIntervention; etc.;

Neurology specialty comprises genes-rendered fundamental medicine-health-healthcare relational data in an among categories: AutonomieDisorders; BrainFluids; Cognition; Coma; CongenitalDiseases; DrugDependencies; Entrapments; GeneticDiseases; Headaches; etc.;

Said genes-rendered fundamental medicine-health-healthcare relational data comprised compilations of relational data of relations of chronologically ordered relational sequences of timestamped data related to diagnoses and related diagnoses and differential diagnoses.

The following is a small sample of relation data in said infinitely immense compilations of said relations of fundamental medicine-health-healthcare relational data of relations of chronologically ordered relational sequences of timestamped data related to diagnoses and related diagnoses and differential diagnoses:

Diagnosis: Coronary Atherosclerosis Risk;

Chief Complaint: Chest pain; neck pain;

Sign: no clinical manifestation; flow-limiting stenoses; ischemia-induced collateral vessels formation; stable effort-induced angina pectoris; demand-induced angina pectoris; acute atherosclerotic syndrome; unstable atherosclerotic syndrome;

Symptom: Chest pain; neck pain; arm pain;

Review of System: Coronary atherosclerosis risk (CAR);

Physical Examination: tendon xanthomas; tuberous xanthomas; palmar xanthomas; tuberoeruptive xanthomas; eruptive xanthomas;

Laboratory: x-rays, CTScan, MRI, ultrasounds; BMI>40 kg/m2; HS-CRP 3.1-10.0 mg/l;

Cigarette smoking; Diabetes; Fasting glucose >110 mg/dl; Random glucose >200 mg/dl; 2-hour glucose >200 mg/dl; Hemoglobin A1C>5.7%;

Linked Diagnosis: Atherosclerosis; Very high CAR; High CAR; Moderately high CAR; Moderate CAR; Low CAR;

Related Diagnosis: Lipid Metabolism; Metabolic syndrome; Lifestyle; Aging; Sedentary; Physical inactivity; Atherogenic diet; diabetes; Hypertensive vascular disease; Blood pressure classification; Coronary heart disease; Acute coronary syndrome;

Differential Diagnosis; C3 cervical radiculopathy; C4 cervical radiculopathy; C5 cervical radiculopathy;

Medical-imagings: MRI 3D coordinates: anatomical areas, sites, parts and organs; CTScan 3D coordinates: anatomical areas, sites, parts and organs; x-ray 3D coordinates: anatomical areas, sites, parts and organs; ultrasound 3D coordinates: anatomical areas, sites, parts and organs; Treatment: lower LDL; anti-fat absorption medications;

Diagnosis: Cervical radiculopathy;

Chief Complaint: Chest pain; neck pain;

Sign: orthostatic hypotension; postural orthostatic tachycardia syndrome; exaggerated orthostatic tachycardia; tachycardia; nausea; vomiting; gastroparesis; constipation; diarrhea; atonic bladder;

Symptom: Chest pain; neck pain; arm pain;

Review of System: dysautonomia; widespread autonomic dysfunctions; cutaneous sensory loss; motor disorders; Guillain-Barre syndrome; AIDS;

Physical Examination: tender trigger point;

Linked Diagnosis: Neck injury;

Differential Diagnosis: acute intermittent porphyria; alcoholic neuropathy; amyloid neuropathy; botulism; diabetic neuropathy; Fabry disease; Guillain-Barre syndrome; paraneoplastic neuropathy; Riley-Day syndrome; familial dysautonomia;

Laboratory: x-rays, CTScan, MRI, ultrasounds;

Medical-imagings: MR 3D coordinates: anatomical areas, sites, parts and organs; CTScan 3D coordinates: anatomical areas, sites, parts and organs; x-ray 3D coordinates: anatomical areas, sites, parts and organs; ultrasound 3D coordinates: anatomical areas, sites, parts and organs; Treatment: rest;

Said networked repositories rendering queries which rendering plurality of interactive, adaptable and changeable multi-dimensional figurines comprising plurality of interactive, adaptable and changeable labeled, accentuated and highlighted, relating to said queries, adaptably attaching and associating plurality of interactive popups comprising interactive, adaptable and changeable queries. Said networked repositories for querying for queries which are diagnoses and related diagnoses and differential diagnoses of said Chief Complaint; Chest pain; neck pain in said interactive and adaptable digital router.

So, said adept networked repositories responding to said interactive adaptable and changeable queries by getting, organizing, analyzing and organizing said queries in ordered sequences of relational timestamped data and scanning, matching and relating said ordered sequences of relational timestamped data with similar, same and distinguishing chronologically ordered sequences of relational timestamped data, in compilations of relations of genes-processor units-rendered fundamental medicine-health-healthcare relational data, rendering adept networked repositories-rendered Interactive and changeable approximates, similars, accurate, match and same as said queries.

Said interactive and adaptable digital router rendering and adapting said interactive and adaptable digital router for rendering said responses, to said networked repositories rendering queries, comprising plurality of interactive, adaptable and changeable multi-dimensional figurines comprising said plurality of interactive, adaptable and changeable labeled, accentuated and highlighted, relating to said queries, adaptably attaching and associating plurality of interactive popups comprising interactive, adaptable and changeable queries.

Said innovative computer programs instructing said processor units to scan, match and relate said queries with similar, same and distinguishing chronologically ordered relational sequences of timestamped data in said compilations of relations of genes-processor units-rendered fundamental medicine-health-healthcare relational data to render scans, adept networked repositories-rendered Interactive and changeable approximates, similars, accurate, match and same as said queries.

Said innovative computer programs instructing said processor units to scan, match and relate said queries to similar, same and distinguishing chronologically ordered relational sequences of timestamped data in compilations of relations of genes-processor units-rendered fundamental medicine-health-healthcare relational data to render diagnoses and related diagnoses and differential diagnoses.

Said adept networked repositories rendering plurality of interactive, adaptable and changeable labeled, accentuated and highlighted, relating to said adept networked repositories-rendered Interactive and changeable approximates, similars, accurate, match and same, adaptably attaching and associating plurality of interactive popups comprising interactive adaptable and changeable adept networked repositories-rendered Interactive and changeable approximates, similars, accurate, match and same and related diagnoses and related diagnoses and differential diagnoses;

said queries and related responses, in and among said networked communication devices for rendering and storing MRI, CTScan, x-ray, ultrasound PET Scan and said adept networked repositories-rendered compilations of relations of said medical-imagings comprising 3D coordinates of anatomical areas, sites, parts and organs in plurality of multi-dimensional anatomical views, illustrations and medical-imagings of digital figurines.

A case in point, using said Coronary Atherosclerosis Risk vs. Cervical radiculopathy both of which share the same Chief Complaint: Chest pain and neck pain, which is almost impossible for said networked repositories for querying for responses. However, further to aforementioned description:

first function for rendering said responses in and among adaptable labeled, accentuated and highlighted multi-dimensional anatomical views, illustrations and medical-imagings of digital figurines wherein said adaptable labeled, accentuated and highlighted adaptably attaching and associating plurality of interactive popups comprising clicking of neck in said interactive, adaptable and changeable multi-dimensional figurines and wherein said queries for autocoordinating and autorouting, showing and rendering interactive and adaptable responses. In this, said adaptable labeled, accentuated and highlighted plurality of multi-dimensional anatomical views, illustrations and medical-imagings of digital figurines and MRI, CTScan, x-ray ultrasound PET Scan adaptably attaching and associating plurality of interactive popups on, for example, the heart, coronary arteries, heart muscles and upper gastrointestinal tract in and among Mm and coordinates, and neck and arm in CTScan and x-ray.

Thus, in said example, a characteristic diagnosis-related labeled, accentuated and highlighted plurality of multi-dimensional anatomical views, illustrations and medical-imagings of digital figurines and MRI, CTScan, x-ray, ultrasound and/or PET Scan adaptably attaching and associating plurality of interactive popups.

However, if said networked repositories for rendering said queries by said networked repositories clicking rhomboideus muscle trigger point of interactive, adaptable and changeable multi-dimensional figurines, further to aforementioned description, said plurality of interactive, adaptable and changeable labeled, accentuated and highlighted and adaptably attaching and associating plurality of interactive popups will change to infra; or said plurality of interactive popups detaching and dissociating from said labeled, accentuated and highlighted.

wherein said queries for autocoordinating and autorouting, showing and rendering interactive and adaptable responses. In this example, said adaptable labeled, accentuated and highlighted plurality of multi-dimensional anatomical views, illustrations and medical-imagings of digital figurines and MRI, CTScan, x-ray, ultrasound and/or PET Scan adaptably attaching and associating plurality of interactive popups on, for example, the neck in said figurines, cervical spine including ligaments, joints, foramina in and among MRI and coordinates of the neck and posterior chest including rhomboideus muscle x-ray.

Thus, in this latter example, yet another characteristic diagnosis-related labeled, accentuated and highlighted plurality of multi-dimensional anatomical views, illustrations and medical-imagings of digital figurines and MRI, CTScan, x-ray, ultrasound and/or PET Scan known herein as iLAH adaptably attaching and associating, or adaptably detaching and dissociating, plurality of interactive popups.

Further to said iLAH, every diagnosis comprised a unique characteristic diagnosis-related labeled, accentuated and highlighted plurality of multi-dimensional anatomical views, illustrations and medical-imagings of digital figurines and MRI, CTScan, x-ray, ultrasound and/or PET Scan adaptably attaching and associating plurality of interactive popups. And, in, between and among said queries for getting said diagnosis and responses, plurality of labeled, accentuated and highlighted multi-dimensional anatomical views, illustrations and medical-imagings comprised said relational data of said queries for getting said diagnosis and responses.

Said second function is well described—reverse deducing responses whereby said adept networked repositories reverse deducing responses to said queries by rendering plurality of interactive, adaptable and changeable labeled, accentuated and highlighted adaptably attaching and associating plurality of interactive popups comprising interactive adaptable and changeable diagnoses and related diagnoses and differential diagnoses and simultaneously rendering plurality of interactive, adaptable and changeable labeled, accentuated and highlighted adaptably attaching and associating plurality of interactive popups comprising said interactive adaptable and changeable genes-processor units-rendered fundamental medicine-health-healthcare relational data comparing said adept networked repositories-rendered adept networked repositories-rendered Interactive and changeable approximates, similars, accurate, match and same;

Further to said description, reverse deducing responses whereby said adept networked repositories reverse deducing responses to said queries by rendering plurality of interactive, adaptable and changeable labeled, accentuated and highlighted adaptably attaching and associating plurality of interactive popups comprising interactive adaptable and changeable diagnoses and related diagnoses and differential diagnoses which simultaneously getting related relational data to rendering related plurality of interactive, adaptable and changeable labeled, accentuated and highlighted adaptably attaching and associating plurality of interactive popups comprising said related relational data.

Said adept networked repositories augmenting said renderings of queries and responses with connected assortments of SIVSF for silhouetting SIUNDV, in and among said viewing screens and touchscreens, adaptably attaching and associating plurality of interactive, adaptable and changeable labeled, accentuated and highlighted and adaptably attaching and associating plurality of interactive popups; and

Said interactive, adaptable and changeable labeled, accentuated and highlighted reversibly detaching to and dissociating from said plurality of interactive popups whenever dissociations consequent to said adept networked repositories dissolving said relations.

Said adept networked repositories rendering plurality of interactive, adaptable and changeable labeled, accentuated and highlighted relating to said adept networked repositories-rendered Interactive and changeable approximates, similars, accurate, match and same.

Said interactive, adaptable and changeable labeled, accentuated and highlighted visual, audio and digital images, representations, illustrations surface anatomical views and related x-ray coordinates, CTScan coordinates, MRI coordinates, ultrasound coordinates in digital anatomy in digital images, representations and illustrations;

Said interactive, adaptable and changeable labeled, accentuated and highlighted adaptably attaching and associating plurality of interactive popups.

Said interactive, adaptable and changeable labeled, accentuated and highlighted adaptably detaching and dissociating plurality of interactive popups; and in connected assortments of SIVSF for silhouetting SIUNDV.

Said connected assortments of SIVSF for silhouetting SIUNDV are interactive adaptable visual-hierarchized constituent flowingframeworks (SIVSF) of and silhouetting said interactive adaptable universal network data-driven visualizations of visual-hierachied flowingframeworks (SIUNDV) (Ser. No. 15/495,164 entitled AIR Oracle).

Said reverse deducing responses whereby said adept networked repositories reverse deducing responses to said queries by rendering plurality of interactive, adaptable and changeable labeled, accentuated and highlighted comprising said interactive, adaptable and changeable labeled, accentuated and highlighted visual, audio and digital images, representations, illustrations surface anatomical views and related x-ray coordinates, CTScan coordinates, MRI coordinates, ultrasound coordinates in digital anatomy in digital images, representations and illustrations; said interactive, adaptable and changeable labeled, accentuated and highlighted adaptably attaching and associating plurality of interactive popups;

said interactive, adaptable and changeable labeled, accentuated and highlighted adaptably detaching and dissociating plurality of interactive popups; and

in connected assortments of SIVSF for silhouetting SIUNDV.

Said plurality of interactive popups comprising process of said adept networked repositories option completing and autocompleting said queries and responses in connected assortments of SIVSF for silhouetting SIUNDV in and among related plurality of interactive, adaptable and changeable labeled, accentuated and highlighted.

Said process of said adept networked repositories option completing and autocompleting said queries and responses comprising accompanying process of toggling popups and related popups in and among related plurality of interactive, adaptable and changeable labeled, accentuated and highlighted.

Said process of said functions and functions-rendered responses adapting, metamorphosizing and transforming said networked repositories said functions and functions-rendered responses rendering genes-rendered dominant chronologically ordered relational sequences of timestamped data including images and videos supplanting corresponding said processor units-rendered queries of said networked repositories for querying.

In other words, genes-rendered dominant chronologically ordered relational sequences of timestamped data including images and videos—related to diagnoses—are well described in said preferred embodiments being rendered said adept networked repositories in and among said networked communication devices.

Said adept networked repositories-rendered compilations of relations of fundamental medicine-health-healthcare relational data comprised diagnoses and diagnoses-specific genes-rendered dominant chronologically ordered relational sequences of timestamped data including images and videos related to diagnoses. Said compilations of relations of fundamental medicine-health-healthcare relational data are well described in prominent textbooks and literatures such as, but not limited to, Harrison's Principles of Internal Medicine. Kasper D L et al. 16th ed. McGraw Hill. There are medical imagings textbooks and literature on the subject of imagings described herein.

In said MRI, CTScan, x-ray, ultrasound PET Scan and said adept networked repositories-rendered compilations of relations of said medical-imagings comprising 3D coordinates of anatomical areas, sites, parts and organs in plurality of multi-dimensional anatomical views, illustrations and medical-imagings of digital figurines, said preferred embodiments comprise, but not limited to, the composition and cataloging said inherent and unique characters according to the claims are the composition and cataloging the inherent and unique surface anatomy gross anatomy, microscopic anatomy, physiological constitution, neuronal innervation including autonomic innervation, afferent innervation, efferent innervation, dermatomal organization, myotomal organization, sclerotomal organization, somatoviscerals, viscerosomatics, neurosomatics, referred pain pattern, myofascial point, motor point, acupuncture point of said specific site within the anatomical boundary defined by the anatomical cleavages and landmarks of said site. In addition, the process also involve identifying adjacent and distant anatomical sites which bear distinct and unique anatomical, physiological, neurological, neuroanatomical, neurophysiological relationship of to said site. In other words more said information as the substrate for said process can be further gathered as needed to fulfill all the needs to produce said nomenclatures, labels, names, symbols and colors of all sites on figurine 1 without departing from the spirit of the invention or the scope of the claims. The result of the process of cataloging is a specific and distinct label expresses as alphabetical, numerical, alphanumeric nomenclature and name of each site expressing said site unique characters and features and said site unique and inherent relationship with adjacent sites and distant sites. The result of said processes is figurine 1 according to the claims wherein said three-dimensional human figurine comprises a three-dimensional coordinate system of named labels within the anatomical boundary defined and delineate by named anatomical cleavages and landmarks of the site represents by said label which is also visualized results in a specific and distinct label express as alphabetical, numerical, alphanumeric nomenclature, panel of symbols such as but not limited to lines, dots, iconic, typographic, mathematical, and alphabetic symbols. In addition a panel of distinct, different and contrast colors shades, tones and any combination thereof can be assigned to each said site base on said cataloguing. Thereby a three-dimensional system of labels base on said processes leading to the origination of nomenclatures, names, symbols and colors on figurine 1 is originated. Said nomenclatures and names are more readily used by persons and nonhuman (henceforth included as person) familiar with technical technology such as computers and physicians. On the other hand, said panel of symbols and colors and any combination of symbols and colors are more readily recognized, better articulated and understood by anyone including laypersons such as any physicians and patients. The emphasis and use of named anatomical cleavages and landmarks on figurine 1 by the present invention facilitate and make possible said communication.

The representation of the substrate which is assigned distinctive and unique alphabetical, numerical and alphanumeric nomenclature, symbol code and color code as discussed supra is as follows:

Said preferred embodiments comprise System of Anatomical Planes. System of Anatomical Landmarks. System of Anatomical Contour and Relief. System of Gross Anatomy by Joints and Ligaments. System of Gross Anatomy of Bursae.

System of Neurophysiologically and Neuroanatomically Related System. System of Dermatomes. System of Acupuncture Points and myofascial pain and trigger points and referred pain pattern. System of Motor Points.

System of Gross and Microscopic Anatomy by Muscle and soft tissue pain and injury site. System of Referred Pain. System of Autonomic Innervation.

Although various preferred embodiments of the present invention have been described, it will be appreciated by and obvious to those skilled in the art and science that adaptations, variations and derivatives of said embodiments are and will be made and achieved for people, animals and machines and objects without departing from the spirit and scope of the present invention.

1. The AIR Oracle Brain being the interactive and adaptable digital router for autocoordinating and autorouting, showing and rendering interactive and adaptable responses (aka responses) to queries of networked repositories for querying, wherein adept networked repositories possessing abiotic intelligence for rendering a function for rendering said responses and said adept networked repositories for rendering a reverse deduction function for autocoordinating and autorouting, showing and rendering responses and responses-related adept networked repositories-rendered Interactive and changeable approximates, similars, accurate, match and same as said queries, wherein said functions and functions-rendered responses for adapting, metamorphosizing and transforming said networked repositories into said adept networked repositories for rendering diagnoses and related diagnoses and differential diagnoses for diagnosing, surveilling, preventing, predicting, extrapolating, controlling and doctoring diseases, disorders and sequela in, between and among networked communication devices, the AIR Oracle Brain comprising:
said abiotic intelligence comprised innovative computer programs including thesaurus software and commercial software for rendering, option completing, autocompleting collectively known as innovative computer programs, said innovative computer programs for instructing said adept networked repositories and said networked repositories for rendering and adapting said interactive and adaptable digital router wherein innovative computer programs for instructing said adept networked repositories and said networked repositories for rendering, option completing, autocompleting interactive and adaptable queries (aka queries) and responses and for toggling said queries and responses in, between and among plurality of interactive, adaptable and changeable popups and plurality of interactive, adaptable and changeable labeled, accentuated and highlighted collectively known herein as iLAH in, between and among of plurality of interactive, adaptable and changeable multi-dimensional figurines said interactive and adaptable digital router for rendering networks of said networked communication devices; and
said networked communication devices comprised networked servers, smartphones, laptops, desktops and medical-imaging devices and equipment comprised viewing screens and touchscreens, cameras, microphones, said adept networked repositories and said networked repositories comprising processor units attached to memories for storing innovative computer programs for interactive and adaptable digital router and adept networked repositories-rendered compilations of relations of fundamental medicine-health-healthcare relational data.

2. The AIR Oracle Brain being the interactive and adaptable digital router according to claim 1 wherein said adept networked repositories for rendering a function for rendering said responses comprising a function for coordinating, routing and showing said queries for getting plurality of interactive, adaptable and changeable multi-dimensional figurines for adaptably attaching and associating, or adaptably detaching and dissociating, plurality of interactive popups comprised said interactive, adaptable and changeable queries for getting and rendering plurality of interactive popups comprised plurality of interactive, adaptable and changeable responses in, between and among said networked communication devices; and
said adept networked repositories for rendering a reverse deduction function for autocoordinating and autorouting, showing and rendering plurality of interactive, adaptable and changeable multi-dimensional figurines for adaptably attaching and associating, or adaptably detaching and dissociating, said plurality of interactive popups comprised responses being interactive, adaptable and changeable approximated, similar, accurate and matched chronologically ordered sequences of relational timestamped data collectively known herein as adept networked repositories-rendered Interactive and changeable same, accurate and match of said queries and requesting for diagnoses and related diagnoses and differential diagnoses related to said adept networked repositories-rendered Interactive and changeable approximates, similars, accurate, match and same in, between and among said networked communication devices.

3. The function for autocoordinating and autorouting, showing and rendering in, between and among said networked communication devices according to claim 2 comprising:
said plurality of interactive, adaptable and changeable multi-dimensional figurines comprised iLAH for adaptably attaching and associating plurality of interactive popups for rendering adaptable and changeable queries for getting, rendering, option completing, autocompleting responses, according to interactive, adaptable and changeable said queries and responses in, between and among said plurality of interactive popups;
said plurality of interactive, adaptable and changeable multi-dimensional figurines comprised iLAH for adaptably detaching and dissociating said plurality of interactive popups, according to another interactive, adaptable and changeable queries for getting, rendering, option completing, autocompleting responses in, between and among said plurality of interactive popups;
said queries and related responses to said queries for toggling said queries and responses in, between and among said plurality of interactive popups;
said queries and related responses to said queries for toggling said queries and responses in, between and among said iLAH; and
said queries and related responses to said queries for toggling said iLAH in, between and among said plurality of interactive, adaptable and changeable multi-dimensional figurines in, between and among networked communication devices based on said networked repositories-rendered and adapted queries comprised data for getting said adept networked repositories-rendered and adapted responses and said adept networked repositories-rendered and adapted responses comprised genes-rendered fundamental medicine-health-healthcare relational data for responding to said queries.

4. The reverse deduction function for autocoordinating and autorouting, showing and rendering in, between and among said networked communication devices according to claim 2 comprising:
said adept networked repositories for rendering a reverse deduction function for autocoordinating and autorouting, showing and rendering plurality of interactive, adaptable and changeable multi-dimensional figurines comprised iLAH for adaptably attaching and associating, or adaptably detaching and dissociating, said plurality of interactive popups comprised interactive adaptable and changeable diagnoses and related diagnoses and differential diagnoses;
said adept networked repositories for simultaneously rendering plurality of comprised iLAH for adaptably attaching and associating plurality of interactive popups comprised adept networked repositories-rendered Interactive and changeable approximates, similars, accurate, match and same;
said queries and related responses to said queries for toggling said queries and responses in, between and among said plurality of interactive popups;
said queries and related responses to said queries for toggling said queries and responses in, between and among said iLAH; and
said queries and related responses to said queries for toggling said iLAH in, between and among said plurality of interactive, adaptable and changeable multi-dimensional figurines in, between and among networked communication devices based on said networked repositories-rendered and adapted queries comprised data for getting said adept networked repositories-rendered and adapted responses and said adept networked repositories-rendered and adapted responses comprised genes-rendered fundamental medicine-health-healthcare relational data for responding to said queries.

5. The plurality of interactive, adaptable and changeable multi-dimensional figurines according to claim 2 comprising:
iLAH for adaptably attaching and associating and adaptably detaching to and dissociating from said plurality of interactive popups according to said interactive, adaptable and changeable queries and responses in and among said plurality of interactive popups.

6. The iLAH according to claim 5 comprising:
said adept networked repositories and networked repositories-rendered interactive, adaptable and changeable labeled, accentuated and highlighted visual, audio and digital multi-dimensional gross, microscopic and digital anatomical images, medical imagings, views, areas, sites, parts, organs representations and illustrations comprised 3D coordinates, according to said interactive, adaptable and changeable popups comprised said queries and responses in, between and among said viewing screens and touchscreens of said networked communication device.

7. The plurality of interactive popups according to claim 6 comprising:
interactive, adaptable and changeable queries (aka queries) for rendering, option completing, autocompleting interactive, adaptable and changeable responses (aka responses) in, between and among iLAH;
queries for toggling responses in, between and among plurality of interactive popups;
queries for toggling responses in plurality of interactive popups in, between and among iLAH;
responses for rendering, option completing, autocompleting queries in, between and among iLAH;
responses for toggling queries in, between and among plurality of interactive popups;
responses for toggling queries in plurality of interactive popups in, between and among iLAH; and
queries and responses for toggling iLAH in, between and among plurality of interactive, adaptable and changeable multi-dimensional figurines in, between and among said networked communication devices.

8. The AIR Oracle Brain according to claim 1 wherein said functions and functions-rendered responses for adapting, metamorphosizing and transforming said networked repositories into said adept networked repositories comprising:
said interactive and adaptable digital router for rendering networks of said networked communication devices comprising adept networked repositories for networking networked repositories for querying,
wherein said abiotic intelligence for instructing adept networked repositories for rendering said function comprised a plurality of interactive, adaptable and changeable multi-dimensional figurines comprised iLAH and interactive and adaptable popups wherein adept networked repositories for getting and rendering, option completing, autocompleting responses and toggling queries and responses and vice versa in, between and among said iLAH and interactive and adaptable popups in, between and among said plurality of interactive, adaptable and changeable multi-dimensional figurines in, between and among said networked communication devices, according to chronologically ordered sequences of relational timestamped data related to iLAH; and
adept networked repositories rendering accurate, match and same, and related diagnoses and differential diagnoses, supplanting queries in networked repositories for querying.

9. In the AIR Oracle Brain, the method of interactive and adaptable digital router autocoordinating and autorouting, showing and rendering responses to queries of querying networked repositories whereby adept networked repositories rendering said responses to said queries and reverse deducing adept networked repositories-rendered Interactive and changeable approximates, similars, accurate, match and same as said queries whereby autocoordinating and autorouting, showing and rendering diagnoses and related diagnoses and differential diagnoses diagnosing, surveilling, preventing, predicting, extrapolating, controlling and doctoring diseases, disorders and sequela in, between and among networked communication devices wherein, consequently, said functions and functions-rendered responses adapting, metamorphosizing and transforming said networked repositories into said adept networked repositories, the method comprising processes of:
innovative computer programs instructing said adept networked repositories and said networked repositories rendering and adapting said interactive and adaptable digital router whereby innovative computer programs instructing said adept networked repositories and said networked repositories rendering, option completing, autocompleting said queries and responses and toggling said queries and responses in, between and among plurality of interactive, adaptable and changeable popups and plurality of iLAH in, between and among of plurality of interactive, adaptable and changeable multi-dimensional figurines;
said interactive and adaptable digital router networking said networked communication devices; and
said networked communication devices comprised networked servers, smartphones, laptops, desktops and medical-imaging devices and equipment comprised viewing screens and touchscreens, cameras, microphones, said adept networked repositories and said networked repositories comprising processor units attached to memories storing innovative computer programs, interactive and adaptable digital router and adept networked repositories-rendered compilations of relations of fundamental medicine-health-healthcare relational data.

10. The method of interactive and adaptable digital router according to claim 9 whereby adept networked repositories autocoordinating and autorouting, showing and rendering said responses to said queries comprising said adept networked repositories rendering plurality of interactive, adaptable and changeable multi-dimensional figurines adaptably attaching and associating, or adaptably detaching and dissociating, plurality of interactive popups comprised said queries and autocoordinating and autorouting, showing and rendering responses comprised adept networked repositories-rendered Interactive and changeable approximates, similars, accurate, match and same and related diagnoses and related diagnoses and differential diagnoses in, between and among said networked communication devices.

11. The process of said adept networked repositories autocoordinating and autorouting, showing and rendering responses according to claim 10 whereby said adept networked repositories rendering plurality of interactive, adaptable and changeable multi-dimensional figurines comprised iLAH adaptably attaching and associating plurality of interactive popups getting, rendering, option completing, autocompleting queries and responses, according to said queries and option completing, autocompleting responses and vice versa in, between and among said plurality of interactive popups;
iLAH adaptably detaching and dissociating said plurality of interactive popups, according to said queries and option completing, autocompleting responses and vice versa in, between and among said plurality of interactive popups;
queries toggling responses in, between and among plurality of interactive popups;
queries toggling responses in, between and among iLAH in, between and among said plurality of interactive, adaptable and changeable multi-dimensional figurines in, between and among networked communication devices;
responses toggling queries in, between and among plurality of interactive popups; and
responses toggling queries in, between and among iLAH in, between and among said plurality of interactive, adaptable and changeable multi-dimensional figurines in, between and among networked communication devices.

12. The method of interactive and adaptable digital router according to claim 9 whereby adept networked repositories reverse deducing adept networked repositories-rendered Interactive and changeable approximates, similars, accurate, match and same as said queries whereby autocoordinating and autorouting, showing and rendering diagnoses and related diagnoses and differential diagnoses comprising said adept networked repositories autocoordinating and autorouting, showing and rendering plurality of interactive, adaptable and changeable multi-dimensional figurines adaptably attaching and associating, or adaptably detaching and dissociating, said plurality of interactive popups comprised said adept networked repositories-rendered Interactive and changeable same, accurate and match and related diagnoses and related diagnoses and differential diagnoses in, between and among said networked communication devices.

13. The process of said adept networked repositories reverse deducing adept networked repositories-rendered Interactive and changeable approximates, similars, accurate, match and same according to claim 12 whereby said adept networked repositories rendering plurality of interactive, adaptable and changeable multi-dimensional figurines comprised iLAH adaptably attaching and associating plurality of interactive popups getting, rendering, option completing, autocompleting queries and responses, according to said queries and option completing, autocompleting responses and vice versa in, between and among said plurality of interactive popups;
iLAH adaptably detaching and dissociating said plurality of interactive popups, according to said queries and option completing, autocompleting responses and vice versa in, between and among said plurality of interactive popups;
queries toggling responses in, between and among plurality of interactive popups;
queries toggling responses in, between and among iLAH in, between and among said plurality of interactive, adaptable and changeable multi-dimensional figurines in, between and among networked communication devices;
responses toggling queries in, between and among plurality of interactive popups; and
responses toggling queries in, between and among iLAH in, between and among said plurality of interactive, adaptable and changeable multi-dimensional figurines in, between and among networked communication devices.

14. The method according to claim 9 whereby said functions and functions-rendered responses adapting, metamorphosizing and transforming said networked repositories into said adept networked repositories comprising:
said interactive and adaptable digital router rendering networks of said networked communication devices comprising adept networked repositories networking networked repositories for querying;
whereby said abiotic intelligence instructing adept networked repositories rendering said function comprised a plurality of interactive, adaptable and changeable multi-dimensional figurines comprised iLAH and interactive and adaptable popups; and
whereby adept networked repositories getting and rendering, option completing, autocompleting responses and toggling queries and responses and vice versa in, between and among said iLAH and interactive and adaptable popups in, between and among said plurality of interactive, adaptable and changeable multi-dimensional figurines in, between and among said networked communication devices comprised networked servers, smartphones, laptops, desktops and medical-imaging devices and equipment.

15. In the AIR Oracle Brain, the method of the interactive and adaptable digital router in said networked communication devices wherein adept networked repositories rendering a plurality of interactive, adaptable and changeable multi-dimensional figurines comprised iLAH and interactive and adaptable popups option completing, autocompleting responses and toggling queries to autocoordinating and autorouting and showing responses to queries and vice versa in, between and among interactive and adaptable popups and iLAH;
and complementary to said function, adept networked repositories reversely deducting by autocoordinating and autorouting and showing a plurality of interactive, adaptable and changeable multi-dimensional figurines comprised iLAH attaching and associating, or detaching and dissociating interactive and adaptable popups comprised interactive and changeable diagnoses and related interactive and adaptable popups comprised diagnoses-related interactive and changeable approximates, similars, accurate, match of and same option completing, autocompleting responses and toggling queries to autocoordinating and autorouting and showing responses to queries and vice versa; and
said functions and functions-rendered responses adapting, metamorphosizing and transforming said networked repositories into said adept networked repositories, the method comprising processes of:
innovative computer programs instructing said adept networked repositories and said networked repositories rendering and adapting said interactive and adaptable digital router whereby innovative computer programs instructing said adept networked repositories and said networked repositories rendering, option completing, autocompleting said queries and responses and toggling said queries and responses in, between and among plurality of interactive, adaptable and changeable popups and plurality of iLAH in, between and among of plurality of interactive, adaptable and changeable multi-dimensional figurines;
said interactive and adaptable digital router networking said networked communication devices; and
said networked communication devices comprised networked servers, smartphones, laptops, desktops and medical-imaging devices and equipment comprised viewing screens and touchscreens, cameras, microphones, said adept networked repositories and said networked repositories comprising processor units attached to memories storing innovative computer programs, interactive and adaptable digital router and adept networked repositories-rendered compilations of relations of fundamental medicine-health-healthcare relational data.

16. The process of said interactive and adaptable digital router autocoordinating and autorouting and showing responses to queries and vice versa, according to claim 15 comprises the processes of:
adept networked repositories getting, organizing, analyzing and organizing queries in ordered sequences of relational timestamped data and scanning, matching and relating said ordered sequences of relational timestamped data with similar, same and distinguishing chronologically ordered sequences of relational timestamped data in compilations of relations of genes-processor units-rendered fundamental medicine-health-healthcare relational data in said memories and rendering interactive and changeable approximates, similars, accurate, match and same as queries;
adept networked repositories rendering, option completing, autocompleting interactive and changeable approximates, similars, accurate, match and same and related interactive and changeable diagnoses in, between and among interactive and adaptable popups adaptably attaching and associating, or adaptably detaching and dissociating, iLAH, according to chronologically ordered sequences of relational timestamped data related to iLAH in, between and among a plurality of interactive, adaptable and changeable multi-dimensional figurines in said networked communication devices.

17. The process of reverse deducting according to claim 15 comprises the processes of:
adept networked repositories getting, organizing, analyzing and organizing queries in ordered sequences of relational timestamped data and scanning, matching and relating said ordered sequences of relational timestamped data with similar, same and distinguishing chronologically ordered sequences of relational timestamped data in compilations of relations of genes-processor units-rendered fundamental medicine-health-healthcare relational data in said memories to rendering interactive and changeable approximates, similars, accurate, match and same as queries;
said interactive and changeable approximates, similars, accurate, match and same option completing, autocompleting interactive and changeable diagnoses in, between and among interactive and adaptable popups adaptably attaching and associating, or adaptably detaching and dissociating, iLAH in, between and among plurality of interactive, adaptable and changeable multi-dimensional figurines in said networked communication devices.

18. The method according to claim 15 whereby said functions said functions and functions-rendered responses adapting, metamorphosizing and transforming said networked repositories into said adept networked repositories comprising:
said interactive and adaptable digital router rendering networks of said networked communication devices comprising adept networked repositories networking networked repositories for querying;
whereby said abiotic intelligence instructing adept networked repositories rendering said function comprised a plurality of interactive, adaptable and changeable multi-dimensional figurines comprised iLAH and interactive and adaptable popups; and
whereby adept networked repositories getting and rendering, option completing, autocompleting responses and toggling queries and responses and vice versa in, between and among said iLAH and interactive and adaptable popups in, between and among said plurality of interactive, adaptable and changeable multi-dimensional figurines in, between and among said networked communication devices comprised networked servers, smartphones, laptops, desktops and medical-imaging devices and equipment, according to chronologically ordered sequences of relational timestamped data related to iLAH.
